(19) United States
(12) Patent Application Publication
(10) Pub. No.: US 2007/0055197 A1
(43) Pub. Date: Mar. 8, 2007

(54) Peritoneal catheter support garment

(76) Inventor: Marie Shakir, Pearland, TX (US)

Correspondence Address: JOHN K. BUCHE, 4445 EASTGATE MALL BLVD., 2ND FLOOR, LA JOLLA, CA, 92121, US

(21) Appl. No.: 11/105,673

(22) Filed: Apr. 15, 2005

(51) Int. Cl. A61M 1/00

(52) U.S. Cl. 604/29

(57) ABSTRACT

A looping support garment for use by peritoneal dialysis patients wherein said garment comfortably covers and secures a peritoneal catheter by an elastomeric means preferably fixed in cuff edges of the garment. The looping support garment and its embodiments are easy to sterilize, may be used in aqueous environments, and are comfortable to the patient who is typically required to take the garment off and on throughout the day.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1. Field of Invention

The present invention relates to the field of peritoneal dialysis and support garments for use with the treatment.

2. Description of Prior Art

Peritoneal dialysis is treatment for kidney failure. When a person's kidneys fail, they cease to effectively clean blood by removing excess fluid, minerals and waste. When kidneys fail, harmful wastes build up in the body and side effects may include hormone irregularity, blood pressure irregularity, anemia and retention of excess fluids. Since the 1980s, peritoneal dialysis (“PD”) has become a more widespread and practical treatment for ailing kidneys. The treatment works by filling a patient's abdomen with dialysis solution through a catheter that inserts directly into the patient's abdomen and through the peritoneum, which is the membrane that lines the wall of the abdominal cavity. The dialysis solution typically contains the sugar dextrose, which solution is adapted to maximize osmotic forces that cause wastes to pass through the peritoneum from the blood and into the dialysis solution. This is the “exchange.” After the exchange, the dialysis solution is then drained from the patient's abdomen, along with all the transferred waste and the patient's blood is cleaned. A patient may undergo three to four treatments per day, depending on the particular treatment regimen. There are a variety of types of PD, which include continuous ambulatory peritoneal dialysis (CAPD), continuous cycler-assisted peritoneal dialysis (CCPD), and nocturnal intermittent peritoneal dialysis (NIPD). The first type, CAPD is the most common and does not require a machine. Instead, the patient uses gravity to fill and empty his or her abdominal cavity with dialysis solution as part of the treatment. All of the various PD treatments, however, share the requirement for a catheter that inserts into the patient's abdomen. The catheter is typically inserted through a small incision, often below or to the side of a patient's navel, and the catheter is guided through the peritoneum into the abdomen. Scar tissue develops around the tube and holds the tube in place, typically on cuffs formed from substances such as polyester fabric that merge with the scar tissue over time. The catheter essentially becomes a fixture to the patient. A typical catheter may extend from the patient's abdomen in the range of 3-14 inches. The catheter is usually made from soft tubing and will wrap around the patients outer abdomen when not in use. The patient is required to maintain the catheter in a meticulously clean state to prevent complications from peritonitis, which is an infection of the peritoneum. Despite all the improvements with PD and changes in design of the catheters, patients are still plagued with the physical reality of a 3-14 inch tube extending from the abdomen. The omnipresent catheter has a tendency to be bulky, it can move around while the patient sleeps, and it gets in the way when a patient performs normal daily activities such a dressing, using the restroom, or even embracing a loved one.

The present inventor has found existing technologies that deal with these problems to be inadequate, primarily because they are excessively complicated, bulky or difficult to use. Almost all of the existing technologies rely on a complicated array of features which include, but are not limited to hoops, belts, buckles, Velcro® fasteners, pouches, loops, pockets, harnesses, pockets, etc. Other patents focused on improving this technology include: Pat. No. 5,496,282 (1996) to Militzer (a belt styled apparatus featuring an array of loops and fasteners supporting the tube on the outside of the belt); Pat. No. 5,688,248 (1997) to Lessing (a belt styled device featuring zippers and buttons); Pat. No. 5,755,698 (1998) to Kagan (a thoracic catheter support in the shape of a harness); Pat. No. 4,533,355 (1985) to Fair (a shorts-styled garment featuring pockets for concealing an ostomy appliance); Pat. No. 4,955,867 (1990) to Endo (a belt styled device with a pouched catheter); Pat. No. 5,853,396 (1998) to Bennes (a belt styled apparatus featuring a catheter pouch); Pat. No. 5,468,229 (1995) to Chandler (a belt styled apparatus featuring fasters on each end, and loops to secure the catheter on the outside surface of the belt); Pat. No. 5,425,719 (1995) to Lessing (a belt pack with a zipper); Pat. No. 5,403,285 (a thoracic catheter support featuring a reclosable belt); Pat. No. 4,738,661 (1988) to Marut (also a belt apparatus featuring fasteners and loops); and Pat. No. 6,126,639 (2000) issued to Sutherland (a similar belt styled device). All of these various features and noble inventions may secure the catheter, but they are extremely complicated to take off and on, particularly considering the frequency required by a patient's treatment regimen. Every additional fastener, loop, or zipper that must be hitched and unhitched becomes a nuisance, and a menacing protrusion that disrupts sleep and normal life activities. The prior art devices can be undesirable and unsightly, for instance if the patient wants to go to a swimming pool, and without advertising to the world his or her peritoneal catheter situated on the outside of a complicated belt or harness. Furthermore, the above captioned inventions present a problem for cleanliness, which is of paramount importance in preventing peritonitis. Fasteners featuring hook and pile closures can be difficult to sterilize and can harbor bacteria. The inventor has solved many of the foregoing problems with a new device and method of using the device.

### SUMMARY OF THE INVENTION

Accordingly, it is the objective of the present invention to provide a novel garment that will simplify the life of peritoneal dialysis patients by providing a more comfortable and concealable device that is easy to take off and on. The garment that is the present invention features a looping construction that circles the abdomen of the patient and covers the protruding peritoneal catheter. The invention does so without the need of a belt-styled apparatus with ends that must be repeatedly fastened and unfastened.

It is a further object of the present invention to provide a support garment that is preferably O-shaped and is adapted to be taken on and off by either stepping into the garment and pulling it up, or by pulling it down over the head and shoulders toward the abdomen, and where the garment is held in place by elastomeric means along its edges.

It is an object of the present invention to provide a garment that is at least as easy to take off and on as elastomeric underwear.

It is a further object of the present invention to provide a garment with minimal features for securing the catheter, so that it is easy to wash and keep clean.

It is a further object of the present invention to provide a garment that is made of cloth, hypoallergenic fabrics, nylons, polyesters, plastics, some of which are water resistant or impervious and may be worn in the shower, while bathing or at the swimming pool.

It is a further object of the present invention to provide a garment that conceals the catheter and secures the catheter underneath the garment, rather than on top or to the outside of the garment. Other objectives of the invention will become apparent to those skilled in the art once the invention has been shown and described.

### BRIEF DESCRIPTION OF THE DRAWING

The manner in which these objectives and other desirable characteristics can be obtained is explained in the following description and attached drawings in which:

FIG. 1 is a perspective view of a one of many prior art devices featuring belt-styled construction to support a catheter on the outside of the garment.

FIG. 2 is a frontal view of the inventor's support garment as worn by the patient over the abdomen so that the garment supports and conceals the PD catheter.

FIG. 3 is perspective view of the looping support garment showing the principal components, namely, the garment material with first and second cuff edges.

It is to be noted, however, that the appended drawings illustrate only typical embodiments of this invention and are therefore not to be considered limiting of its scope, for the invention may admit to other equally effective embodiments.

### DETAILED DESCRIPTION OF THE INVENTION

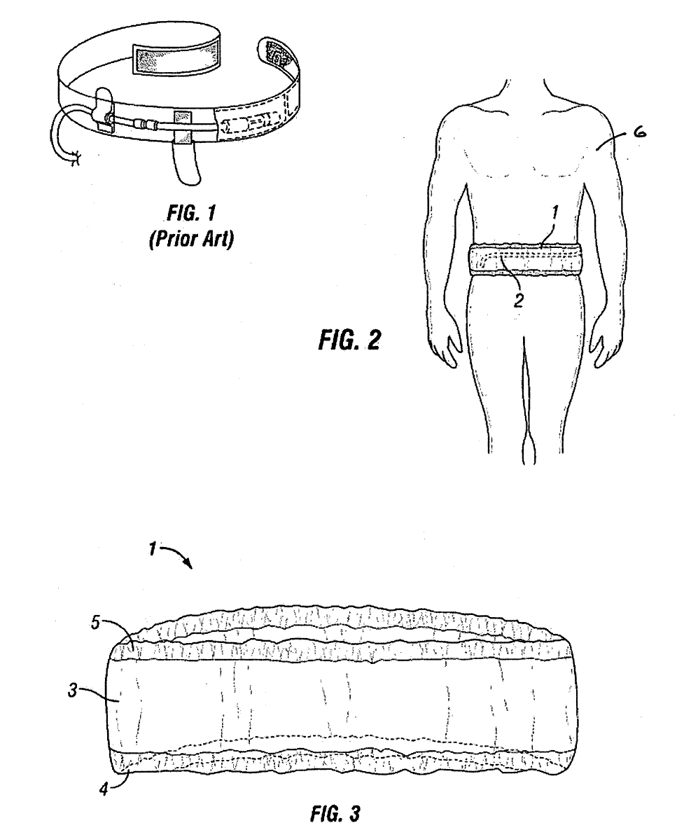

FIG. 1 is a perspective view of the prior art, found in Pat. No. 5,496,282 (1996), featuring an alternate means of supporting a peritoneal catheter on a patient. As is shown in the specification of the patent, the catheter is held in place primarily on the outside of the belt-styled apparatus by an array of fasteners, belts and loops. The prior art effectively secures a patient's catheter, however, provides a relatively rigid securing means which can be uncomfortable for a patient when the device is worn throughout the day and night. Also, the prior art has a variety of surfaces, some of which including hook and pile (Velcro™) styled fasteners, which may cause discomfort on the patient and may be difficult to keep as sanitary as is useful in PD where infection is a constant concern.

FIG. 2 is a frontal perspective view of the present invention, the looping support garment 1, as it is typically used by a patient 6 to support a peritoneal catheter 2, a device by and through which fluids are administered during peritoneal dialysis (“PD”) treatments. The use of peritoneal catheters is well known in the PD field. The catheter 2 is typically permanently secured on a patient 6 in the vicinity of the abdomen and extends from the point of exit on the patient in the typical range of 3-15 inches, although longer or shorter catheters may be employed. The length of the protruding catheter 2 is what creates obstruction and inconvenience to the patient 6, and is a principal problem that this invention helps to alleviate. As shown, the looping support garment 1 slides on or off of the patient 6 and covers the catheter 2 extending from the patient 6. One method of using the looping support garment 1 is to pull the garment up and over the legs to cover the catheter 2. Another method of using the looping support garment 1 is by pulling the garment down over the patient's head and shoulders to where it covers the catheter 2. Because the looping support garment 1 covers the catheter 2, there is no need for loops, buckles or straps to secure the catheter 2 on the outside of the device, as is common in prior art devices. The looping support garment 1 employs elastomeric means for staying in place on the patient's abdomen, with the same elastomeric means serving to confine the catheter 2 underneath the support garment 1, yet without being too rigidly or uncomfortably pressed against the patient's body. Also, the elastomeric means (typically elastic bands or elastic strips fixed or sewn into the garment) permit securing the looping support garment 1 to the patient's body, without the need for belt-styled closing devices, e.g., buckles, loop and pile fasteners, hooks, etc.

FIG. 3 is a frontal perspective view of the looping support garment 1, which shows its construction in greater detail. The invention features a garment material 3, which is formed into the shape of a loop. This may be accomplished by sewing or other suitable means employed in the fabric and apparel industry. The garment material 3 is typically of a width in the range of one to ten (1-10) inches, with the looping length of the garment material 3 made to accommodate various patients and their waist sizes. The sizes are those typically employed in the clothing and apparel industry to designate various sizes of persons from small children to exceptionally large patients, for example, XS, S, M, L, XL, XXL, and XXXL (ranging from 10 to 100 inch circumference). One side of the garment material 3 forms a first cuff edge 4, and the opposite side of the garment material 3 forms a second cuff edge 5. The cuff edges 4 and 5 are preferably of equal widths and house elastomeric means which permit the entire looping support garment 1 to readily contract, expand and secure the catheter 2 that extends from the patient between the two respective cuff edges (4 and 5) and underneath the garment material 3. The elastomeric means may also form the cuff edge, but are preferably sewn inside cuff edges (4 and 5), which form seams, typically in the range of 1 to 3 inches in width. In this manner, the elastomeric means are not in direct contact with the patient's skin. The elastomeric means contained in the cuff edges (4 and 5) allow the user of the looping support garment 1 to take the device on and off with incredible ease, and without needing to fasten and unfasten multiple fasteners. The invention is also easy to clean, which is of the utmost importance in PD treatment and to avoid complications from peritonitis. The invention can be thrown straight into a washer and dryer and the relative simplicity of the surface area of the invention facilitates thorough cleaning. The garment material 3 may be formed of a variety of fabrics known in the apparel industry, including but not limited to cloth, denim, plastic, nylon, vinyl, polyester, blends, cotton, wool, linens, spandex, hypoallergenic fabrics, leather, Rayon, silk, quick-dry fabrics, and a variety of fabrics of the variety sold under GoreTex® trademark. The garment material 3 may be made of fabrics which resist or that are impervious to water. An important preferable embodiment of the present invention is that the looping support garment 1 can be used in the shower, bath or swimming pool. Another notable beneficial feature of the invention is that it conceals the catheter 2 of the patient more effectively than the prior arts, which typically would fasten some portion of the catheter 2 to the outside of the device. The invention may also be formed of printed or unprinted fabrics that will suit the style preferences of the patient 6.

It is to be noted, however, that the appended drawings illustrate only typical embodiments of this invention and are therefore not to be considered limiting of its scope, for the invention may admit to other equally effective embodiments.

I claim:

1. A looping support garment comprising:
(a) a garment material formed into a loop;
(b) said garment material forming a first cuff edge on one side and a second cuff edge on an opposite side;
(c) wherein said first and second cuff edges are adapted to expand and contract;
(d) whereby said looping support garment may be worn by a patient to cover and secure a catheter.

2. The looping support garment of claim 1, wherein said first and second cuff edges house an elastomeric means that permits said looping support garment to expand and contract.

3. The looping support garment of claim 1, wherein said garment material is formed from any of the group of fabrics consisting of either cloth, denim, plastic, nylon, vinyl, polyester, blends of polyester and cotton, cotton, wool, linens, hypoallergenic fabrics, leather, Rayon, silk, quick-dry fabrics, and fabrics sold under GoreTex® trademark.

4. The looping support garment of claim 1, wherein said garment is formed from fabrics that are water resistant.

5. The support garment of claim 1, wherein said garment is formed of fabrics that are impervious to water.

6. The support garment of claim 1, wherein said garment material forms a loop whose circumference measures in the range of ten to one-hundred inches.

7. A method of using a looping support garment in connection with peritoneal dialysis treatment comprising the steps of placing the feet of a patient so that said looping support garment forms a loop around the feet; pulling said looping support garment over the feet, legs and waist; and securing said looping support garment securely over a peritoneal catheter extending from said patient's abdomen.

8. A method of using a looping support garment in connection with peritoneal dialysis treatment comprising the steps of placing said looping support garment over the head so the garment forms a loop around the head; pulling said looping support garment down over the head, shoulders, and arms; and securing said looping support garment securely over a peritoneal catheter extending from said patient's abdomen.
